(19) United States
(12) Patent Application Publication
(10) Pub. No.: US 2016/0192994 A1
(43) Pub. Date: Jul. 7, 2016

(54) AEMS Compendium

(71) Applicant: David Russell, Oklahoma City, OK (US)

(72) Inventor: David Russell, Oklahoma City, OK (US)

(21) Appl. No.: 14/544,443

(22) Filed: Jan. 7, 2015

(51) Int. Cl. A61B 34/20; A61M 5/142; A61B 17/00

(52) U.S. Cl. CPC A61B 34/20; A61B 17/00234; A61M 5/14276; A61B 2034/2068; A61B 2017/00221; A61B 2017/003; A61M 2202/0007; A61M 2205/04

(57) ABSTRACT

Disclosed is an ultraminiaturized Auto-Locomotive Device (ALD) apparatus with tool actuators and traction, locomotion, and propulsion mechanisms. The ALD is capable of static and dynamic activity, including moving to a target work area or structure, stopping, turning, anchoring, operating ALD actuator tools, auxiliary peripherals, etc., in response to external tactical control commands and/or pre-programmed instructions issued by administrative system(s), and/or “expert system(s)” such as enhanced surgery systems and/or medical robotic systems. The ALD is a specialized version of an Array Element Mesh System (AEMS) adapted for precision control and precision tasks such as in-vitro and in-vivo micromanipulation, microsurgery, transportation of organic and inorganic structures and materials; inter- and intra-cellular navigation, locomotion, and propulsion; surgical procedures and operations; and other very-small tasks. Methods and systems for controlling ALD maneuvers and operational tasks are also disclosed. The present invention is typically used for very-small-geometry, micro-electromechanically-executable tasks; cellular-scale surgery; other microscopic techniques; etc.

### CROSS-REFERENCE TO RELATED APPLICATIONS

This Non-Provisional Continuation-In-Part Application provided herein claims priority to application Ser. No. 11/649,898 filed Jan. 3, 2007, which claimed priority to App. Ser. No. 60/755,589 filed Dec. 31, 2005 entitled, “Array Element Mesh System with Deployable Mini-traction and Locomotion Devices for Medical, Maintenance, and other Small Geometry Applications”. Additionally, this CIP Application is related to—and is also a Continuation-in-Part of—U.S. patent application Ser. No. 11/208,250, “Array Mesh Apparatus, System & Methods” filed Aug. 19, 2005, all of which are included by reference in their entirety.

### STATEMENT REGARDING FEDERALLY SPONSORED R & D

There has been no federally-sponsored research and development associated with this instant application and series of applications. There has been no federal subsidy or involvement with these applications, whatsoever.

### BACKGROUND OF THE INVENTION

1. Field of the Invention

The underlying field of the invention is automatic sampling, simulation, and replication of 3D objects, through sampling and robotic technology as implemented in earlier-disclosed Array Element Mesh Systems-based (AEMS-type) products. In the present invention, AEMS products are further adapted for medical applications and other very-small form-factor applications. More particularly, AEMS-related inventions disclosed herein can (variously) sample, simulate, replicate, or manipulate within target work areas or structures to do work. In operation, advanced (application-specific, variously-adapted) AEMS devices can be precisely controlled—(e.g., by external commands and/or by autonomous reconfiguration via any AEMS' own preprogrammed instructions) to “on-the-fly” or autonomously change (a) their physical shape(s), and/or (b) their activity configuration(s), and/or (c) their attached peripherals—to expedite micro-manipulation, micro-navigation, micro-locomotion, and/or microsurgery.

Additionally the field of the invention is transportation of organic and inorganic materials, e.g., in-vivo or in-vitro supply or removal of organic cellular material (or other material) for inter- and intra-cellular surgeries, microscopic operations, or other ameliorations medical and non-medical.

2. Related Art

Recent Innovative Medical Technology Products

While it has been recently proved feasible to swallow a “surgeon device” for use in the mouth, esophagus, stomach, intestines, etc., there remain many unresolved challenges in the area of intelligent medical devices or robotic devices operating on and/or within animate subjects in-vivo, such as animal or human bodies, or even much smaller targets of interest.

In future surgical or in-vivo or in-vitro activities, such devices will increasingly be introduced at any reasonably-accessible entry point. What we do not see in related art, is technology for precise control, navigation, and locomotion of a device, e.g., to a site in (or outside) the body where surgery and/or drug delivery is needed to perform surgical procedures, excise and remove pathology, insert ameliorates, supply prescription medication, etc.

By contrast, the present invention disclosed herein and my expected subsequent disclosures provide precision-control unavailable in other products known to the inventor. As additional background, parent application Ser. No. 11/208,250 in combination with its daughter application Ser. No. 11/649,898 provide a basis for underlying AEMS technology. In this CIP application, AEMS technology is further disclosed and adapted for precision control and product modularity.

Illustrative Medical Applications and Other High-Potential Concepts

TV Camera In-Vivo

Regarding in-vivo medical ameliorative therapies, there've been many ambitious endeavors to improve the human condition and address health challenges. For example, researchers created miniature TV cameras and transmitters in the form of a pill orally introduced into the body. Doctors can then receive transmissions of pictures of structures within the body, as the TV camera moves through the body. There are other in-vivo treatments involving the use of television technology, such as flexible sigmoidoscopy.

Balloon Angioplasty In-Vivo

So-called “Balloon Angioplasty” products having very-small-geometry form factors are routinely used to clear arteries and veins near the heart of plaque/debris. Additionally there are other medical ameliorative devices which can be introduced into the human body, such as stents, screws for supporting weak or broken bones, and artificial medical appliance inserts.

Concepts for Other Advanced In-Vivo Devices

In arguably analogous art, Professor Ludwig Bartels has created a new molecule called 9, 10-dithioanthracene (DTA), which can “walk” across a substrate, such as copper, in a straight line. [Phil Schewe and Ben Stein, Walking Molecules, Physics News Update, Number 751 #2, Oct. 26, 2005].

The DTA molecule has two “feet” that are configured in such a way that only one “foot” may rest on the underlying substrate at a time. When the molecule is activated, it will pull up one “foot” and place the other “foot” down, and in this manner can move forward in a perfectly straight line without requiring preset nano-tracks. The molecule can be activated by heat or physical contact, such as a push by the tip of a scanning tunneling microscope.

The DTA molecule is intended for use in information storage or computation. Due to the chemical structure of DTA, the molecule can only travel in a straight line, and does not respond to navigational commands.

Necessity of the Invention & Unsatisfied Needs

Medical Technology Today

Researchers have succeeded at creating very small form factor in-vivo and in-vitro exploratory and ameliorative medical devices. Such devices are gaining sophistication. Demand is growing for viable products. These technologies enable doctors to view internal workings of human bodies, unobstruct veins and arteries, reconstruct broken bones, etc., and do many other tasks or procedures.

“Precision” Limitations in Medical Technology

Despite success on many product fronts in medical technology serving many major medical applications, it appears few or no products allow an observer (e.g., doctor, technician) to control micro- and nano-scale movement or surgical procedures in-vivo, using a device which is physically reconfigurable “on the fly” on the micro- or nano-scale.

Instantaneous Reconfigurability

The art also appears to lack devices which can alter or transform their physical configuration “on-the-fly” and “as-needed” (e.g., while operating on micro- or nano-scale biostructures; while transporting biomaterial or medication to target biostructures; or while supplying biostructures with medication to ameliorate or fix a target condition, etc.). The art does not appear to contain any precisely-controllable, shape-altering devices that can transport cells and assist the body to work on, ameliorate, or reconstruct itself.

Flexibility, Transport, Delivery

Additionally, the art is very limited with respect to devices capable of introducing ex-vivo materials in-vivo, with the exception of hypodermic needles, catheters, and other well-known, extant devices now being implanted and deployed inside the human body.

Target Micro Manipulation and Other Small-Scale Procedures

There's an unsatisfied need for devices capable of acting, manipulating, and/or ameliorating very-small-scale structures or cell-sized materials in-vivo, to help the body to reconstruct or heal itself. For example, such a device could be used to address internal medical conditions, including the precision repair or replacement of damaged neural pathways in the brain. As a case in point, it has recently been demonstrated and photographed, that intercellular communication between neural cells and between immune cells in the human body can be effectuated via intercellular “nanotubes” composed of cellular membrane. (See: Intrigue at the Immune Synapse, Scientific American, February 2006, p. 55).

Such an “engineered” pathway (artificially-delivered or artificially-built) could be the physical route and/or dead-reckoning track used by a precision-navigating device which would “morph” or re-configure itself (e.g., from a relatively short, wide device into, e.g., an elongated, serpentine 3D object) to adapt itself for travel from neural cell to neural cell, or travel from immune cell to immune cell, or build communications pathways, or any other feasible micro- or nano-scale task.

Accordingly, it appears there's an unsatisfied need in the art for “intelligent”, malleable, flexible, reconfigurable devices capable of introducing ex-vivo materials in-vivo and vice-versa, as well as other necessary or earlier-undoable work, limited only by the skill and experience of the doctor or technician

### SUMMARY OF THE INVENTION

### Advantages of the Invention

The AEMS is a mathematically-precise device able to track and/or plot its' steps. Depending on features deployed in any particular version, it can “morph” (metamorphose, or adapt) itself to move and pass through restricted places. These can include organic or inorganic structures, e.g., cell walls and cellular organisms, arteries, veins, or other organic or inorganic targets (e.g., small membranes or cell-like structures; nanotubes) based on configuration and purpose. Precision control aspects include morphing as implementable, dead reckoning features, and “re-morphability” (based on application) makes multiple, various-function versions of the invention possible. To illustrate: in reproductive medicine, the invention could be deployed to improve fertility and increase probability of normal conception. This device could reduce need for costlier in-vitro solutions and/or preclude or limit use of controversial pharmaceuticals. There's also demand for a version of the device for dermatological applications: the device could be used for topically-applied formulations or rejuvenates or innovative cosmetics, e.g., to aid skin to regenerate itself and eliminate wrinkles, pitting, and other skin conditions caused by sun damage and aging.

Another example of an area where latent demand exists for this invention is in amelioration of myocardial infarction. In the highly-variable range of extent of the infarction, damage of greater or lesser extent is manifest in an area of scarred heart tissue (NB: infarction tissue or dead cells are one casualty of myocardial infarction. My device could replace and/or export the infarction tissue, or do other delicate work. There are many surgical instruments presently being designed illustrating the state of the art.

### OVERVIEW OF FIGURES & REFERENCE NUMERALS

### Figures

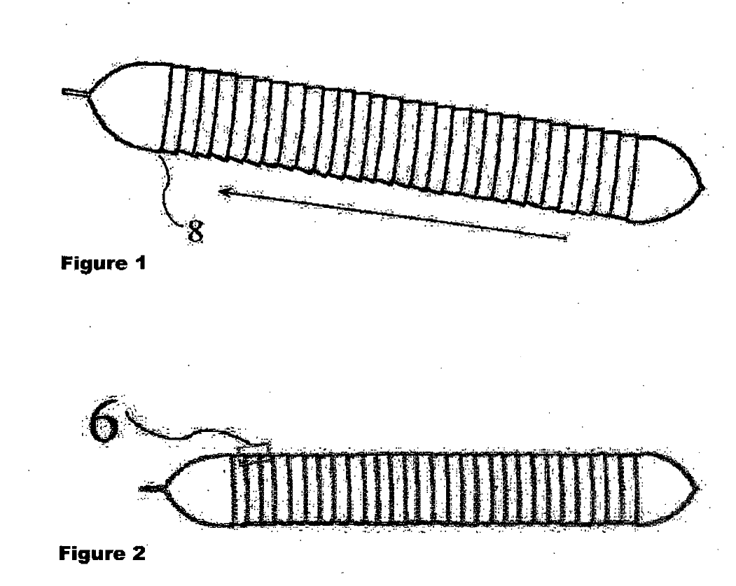

FIG. 1, AEMS with directional friction

FIG. 2, Magnified area outline

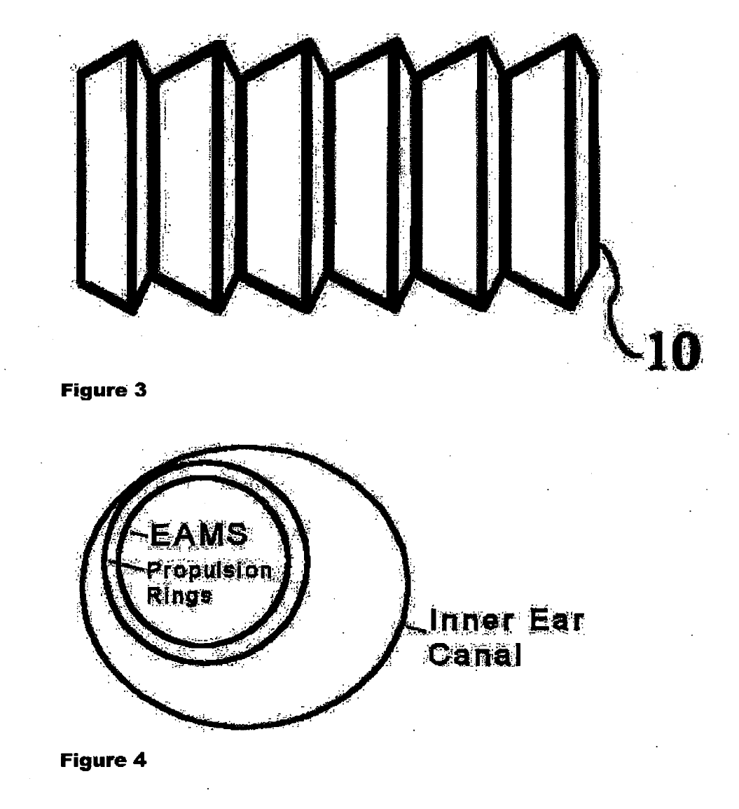

FIG. 3, AEMS with biocompatible shell

FIG. 4, AEMS beginning transport by magnetic oscillation down outer ear canal

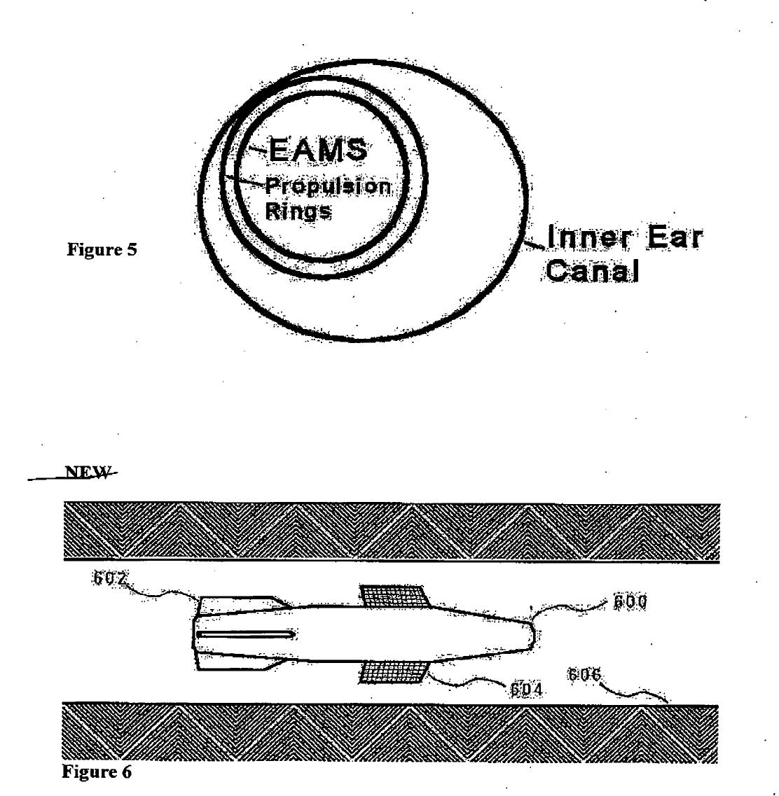

FIG. 5, AEMS proceeding by magnetic oscillation further down outer ear canal

FIG. 6, AEMS Device within an Artery Wall

FIG. 7, AEMS Device (standalone)

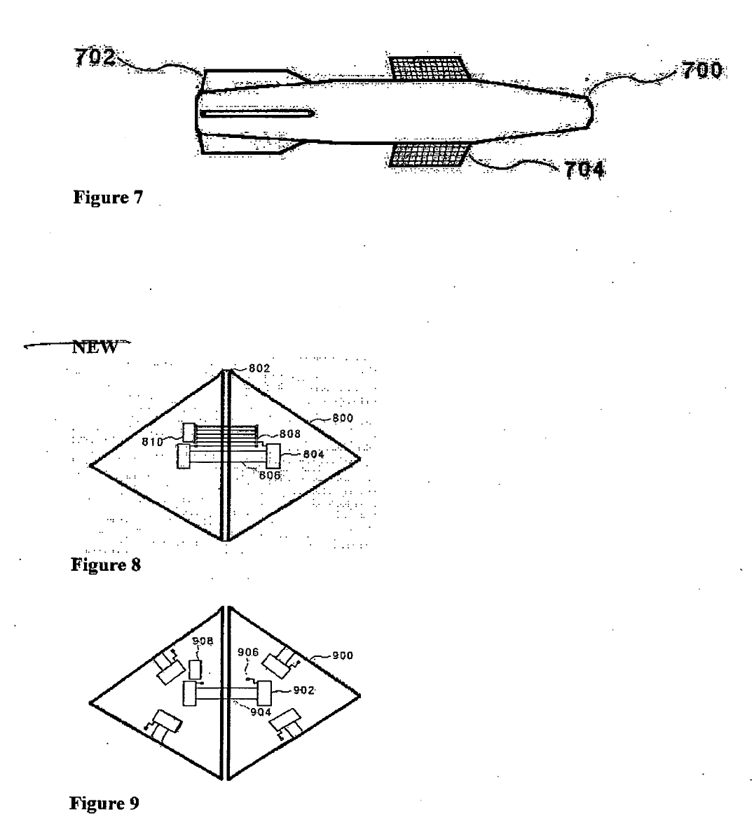

FIG. 8, Joints & Actuator between Two Triangular Array Mesh Element segments

FIG. 9, Top Layer and Bottom Layer separated by Two Triangular Stiffeners/Electro-active, electro-constrictive actuators

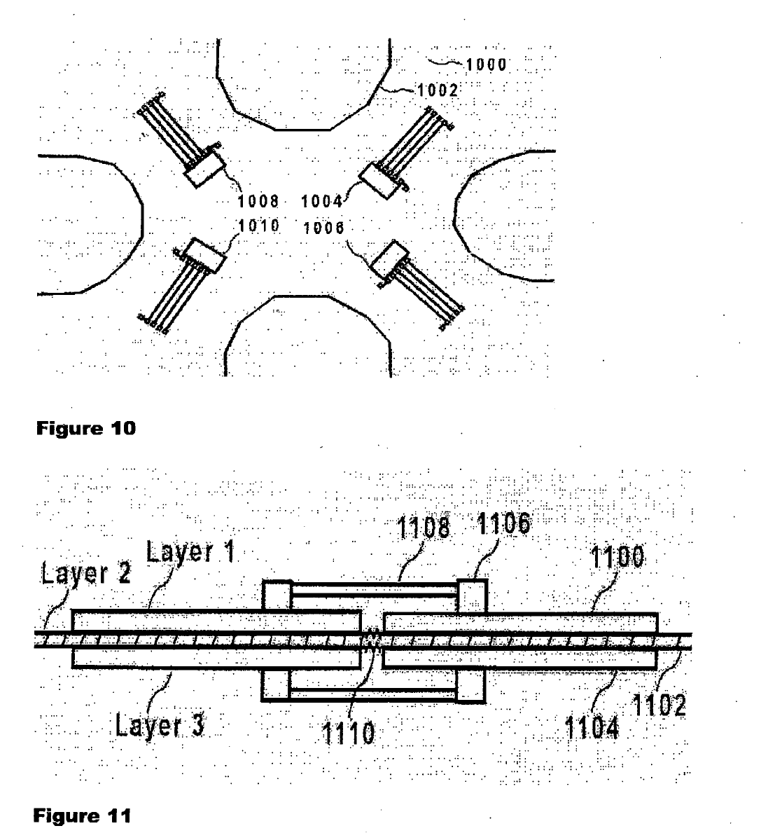

FIG. 10, Middle Layer (2) Flexible Circuit Underlying the Entire Array Mesh

FIG. 11, Edge View, shows 3-Layer Construction of a double-triangular segment embodiment of an AEMS

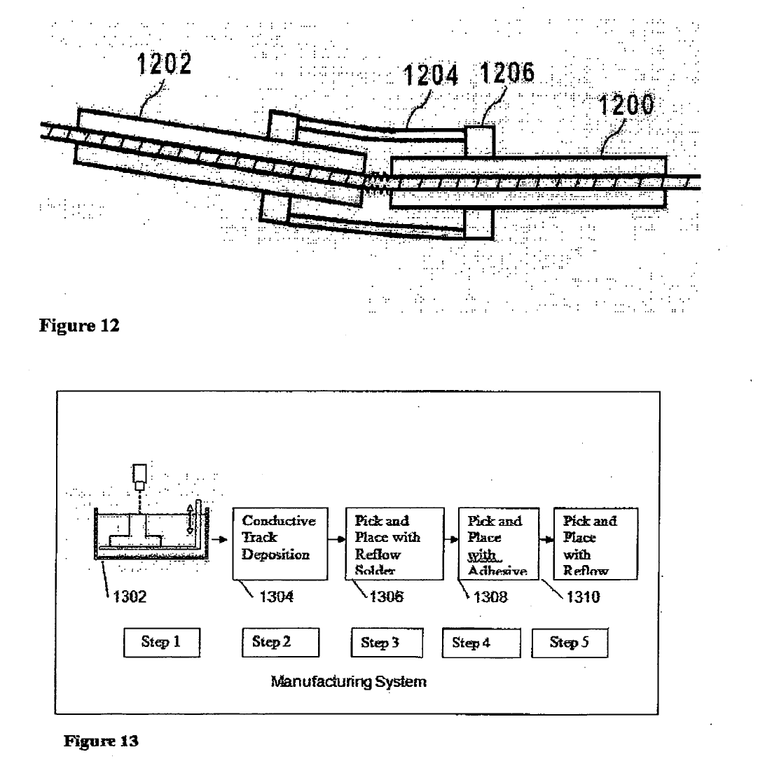

FIG. 12, movable joint, joint being engaged as an ON/OFF switch

FIG. 13, manufacturing process (one preferred embodiment)

### FIGURES WITH REFERENCE NUMERALS

FIG. 1, the basic apparatus (one preferred embodiment), numbered as 8

FIG. 2, Magnified area outlined on basic apparatus invention, numbered as 6

FIG. 3, Magnified area, detail, numbered as 10

FIG. 4, apparatus in transit

FIG. 5, apparatus in further transport

FIG. 6

600 AEMS Device within an Artery Wall

602 Rear Fins of AEMS Device for Vertical and Horizontal Stabilization

604 Array Mesh fins for Propulsion and Control

606 Artery Wall & adjacent tissue (cross-hatched for clarity

FIG. 7

700 AEMS Device itself

702 Rear Fins for Vertical & Horizontal Stability of AEMS

704 Array Mesh fins for Propulsion and Control

FIG. 8: Joints & Actuator between two interconnected triangular Array Mesh Element segments; four more joints, each can be interconnected as specified into each of up to four adjacent Array Elements

800 Array Element Mesh (flex circuit, control chip, electro-constrictive actuator)

802 Middle-layer Flex Circuit interconnectors spanning the gap between doubled interconnected triangular plastic stiffeners/AEMS segments

804 Two Mounting Blocks (at both ends of an electro-constrictive Actuator) affixed onto/over the top of doubled, interconnected triangular stiffeners

806 Top layer electro-constrictive Actuator: a separate bottom layer electro-constrictive Actuator is directly underneath (not shown)

808 Conductive traces on interconnector “Flex Circuit” providing electrical power and communications between two Array Element segments

810 Microprocessor Controller Die, COB-Mounted (Chip On Board) within recess in one triangular pair

FIG. 9: Top Layer & Bottom Layer w/triangular stiffener & electro-constrictive actuator w/four joint interconnector ports for connecting to 4 Adjacent Elements

900 Top and Bottom Layers—two Array Mesh Element half-segments (double triangular segments) with electro-constrictive actuator 904

902 Two Mounting Blocks affixed onto/over on top of the double-triangular plastic stiffeners. There are 6 on this Element Pair

904 Two Mounting Blocks (at both ends of an electro-constrictive Actuator) which are mounted atop this layer and affixed to Mounting Blocks 902

906 bilaterally attachable at end connection from End of Actuator to Flex Circuit On Layer 2, Not Shown on This Top Layer Drawing

908 Recess in one triangular array element segment to accommodate insertion of a Microprocessor or microcontroller Die (a bare die)

FIG. 10: Middle Layer (2) Flexible Circuit, underlies the Entire Array Mesh

1000 Thin, Flexible Circuit extends throughout the Array Mesh System and acts as hinges at the Joints

1002 Relief Holes Punched at Apex of Triangles (allows 3D Flexibility to array)

1004 Processor for Array Network Communications and Controlling the Actuators on Layers 1 and 3

FIG. 11: Edge View shows 3-Layer Construction (Triangular Embodiment)

1100 Layer 1, One of Two Array Mesh Element Stiffeners Cemented to the Top Actuator Mount 1106 and Flex Circuit 1100

1102 Layer 2, Thin Flexible Circuit Which Extends throughout the Array Mesh System and Acts as Hinges at all Joints (crosshatched)

1104 Layer 3, One of Two Array Mesh Element Stiffeners affixed to the bottom actuator mounting and its “Flex Circuit” 1100

1106 uP for array network communications & control of Layers 1 & 3 actuators

1108 One (of 2) electro-constricting actuators; functions as a “muscle” to “flex” and move the Joint up or down

1110 Waves Stamped in the Flexible Circuit Improve 3D Flexing at Multiple Non-collinear hinge points, allowing Joint compression, flexion, and extension

FIG. 12, actuator/joint mobility option: 1202, 1204, 1206, 1208 are the associated movable/moving parts

FIG. 13, manufacturing process with platform and steps

### DETAILED DESCRIPTION OF THE INVENTION'S FIGURES

In FIG. 1 is shown view of a basic apparatus in a streamlined, aggregated, right-cylindrical form. The apparatus deploys in a primarily favoring “one-way” traveling orientation (here, “right-to-left” & counterclockwise). The apparatus shown moves most easily in a (relative) leftward-travelling direction as illustrated. Note that one or more zero-state AEMSs can be folded and/or cut and/or pasted into one or more other AEMSs and/or onto multiple otherwise-configured (differently oriented, printed, folded, cut, doubled, etc.).

FIG. 2 shows an area outlined for a closer-up view (magnification) as indicated by outline window 6 . . . that area is further shown magnified in FIG. 3, infra.

FIG. 3 shows the magnified area of a right cylindrical AEMS outlined in window 6 in FIG. 2. In FIG. 3, a magnified segment 10 of the device is illustrated including multiple individually-collapsible stages. It is most easily leftward-movable, due to its “leftward-streamlining”, built using (1) an application-specific (custom or semi-custom) design (i.e., a prefolded/preconfigured AEMS substrate arrangement) and/or (2) off-the-shelf AEMS substrate (e.g., an EAP), here a rolled, self-connected differentiated or crafted “fabric” or other specific AEMS substrate implementation (e.g., formed here from a series of pre-set “zero state” AEMS and/or a partially-zero-state with part preconfiguration/config “on-the fly”.)

FIG. 4 shows a simplified representation of an extended right cylindrical AEMS device (as in FIG. 1) having multiple “propulsion rings”, depicted in motion “downward” (“away from” the viewer) along an imaginary z-axis, through three dimensions and traveling inside a body cavity representing an inner ear canal.

FIG. 5 continues the movement in FIG. 4, in this second representation of an extended right cylindrical AEMS device proceeding further/deeper into an inner ear canal, by means of repeated use and deployment of moving “propulsion rings” which here cause the device to continue forward motion in most favorable direction as implemented in streamlining.

FIG. 6 depicts an AEMS device 600 deployed within an artery wall 606 which comprises an in-vivo lumen for carrying blood within a mammalian body. Here, a preformed application-specific version of an AEMS device of the invention has a streamlined design favoring movement of the device 600 in the direction of the arterial current. The device shown implements vertical and horizontal stabilizing “rear fins” 602, and also AEMS propeller fins 604 for propulsion and control and for “directional sails”.

FIG. 7 shows an undeployed device 700 alone (equivalent to device 600 in FIG. 6) by itself. Device 700 also provides two sets of fins 702 and 704, each analogous in function to fins 602 and 604.

FIG. 8 depicts joints and an actuator spanning between two (2) interconnected triangular Array Mesh Element segments . . . middle-layer flex circuit 802 connects the doubled interconnected AEMS segments. The flex circuit 802 is comprised of, e.g., a flexible electro-active polymer (EAP) substrate that acts as an ON/OFF switch, connecting the variously-movable triangular plastic stiffeners, making or breaking the switch either ON or OFF. Two (2) mounting blocks 804 (at each end of an electro-constrictive actuator) are affixed onto and over the top (or alternately, under the bottom) of the doubled interconnected triangular stiffeners.

“Top” layer 806 comprises an electro-active, electro-constrictive switch, an ON/OFF actuator in a flex circuit (made of, e.g., an electro-active polymer/EAP substrate). A separate “bottom” layer (not shown) comprises the opposite side of this electro-constrictive Actuator. The bottom layer (not shown) is directly underneath top layer 806. A further integral component, conductive traces 808 on interconnecting flex circuit, provides continuity, electrical power, and communications between these two (2) connected Array Element segments, primary subsystems of the AEMS design. Microprocessor/Controller Die 810, is a COB (Chip On Board) mounted within a recess in one side of the doubled triangular pair segments, comprising one preferred embodiment of each Array Element. In this implementation, die 810 is a “bare die”.

FIG. 9: Top Layer & Bottom Layer 900 (here, half-Element segments on one preferred embodiment of an Array Element) with triangular stiffeners and an electro-active, electro-constrictive actuator also implements four (4) interconnector ports, allowing interconnection thereto of up to four (4) Adjacent Elements (one port serves each connected Element). Here, four (4) more adjacent joints can be provisioned as configured; each further-provisioned joint can be interconnected similarly to the initial joint shown connected at 802 (connecting the first two doubled triangular element pair segments.

As earlier exemplified in FIG. 8 . . . my FIG. 9's two (2) Array Mesh Element half-segments (doubled connected triangular element-segments), are connected together forming one (1) Array Element. Also here is used an electro-constrictive actuator, a “switch”-type flex connector (e.g., an electro-active polymer/EAP). Here, two (2) Mounting Blocks 904 are affixed onto/within or over interconnected double-triangular plastic stiffeners are connected by flex circuit 902. Top layer 906 is bilaterally attached to blocks 904. Microprocessor or microcontroller 908 is affixed onto or into a recess at 908 in one triangular array element segment capable of properly deploying a bare die.

FIG. 10 shows a portion of the array with four joints as an example, although there could be six, eight or more. 1000 is the substrate and 1002 is one of four cutouts shown to provide flexibility. 1004 through 1010 represent the magneto-strictive actuators and position sensors of 1108 on FIG. 11, below.

FIG. 11 describes the actuator joint 1110 with mounting blocks 1106 and magneto-strictive actuators 1108 mounted on the stiffener forms 1100 on array substrate 1102.

FIG. 12 is the same actuator joint as FIG. 11 but activated with actuator 1204 constricting and the joint bending upward as a consequence.

FIG. 13, describes an example of basic manufacturing steps employed to produce the Array Mesh. Since the array can be microscopic, the manufacturing process is automated to handle very small components.

A micro-stereolithographic printer or molding machine 1302 is used to produce a sheet 1000 of FIG. 10 with raised mounting blocks such as 1106 and stiffening portions as described in FIG. 11. This sheet is printed or molded thin in the region that forms joints that bend to enable array mesh motion as shown in 1110 FIG. 11. In the second step 1304 additive conductive tracks are deposited by existing deposition equipment to provide a circuit that accommodates the varying surface levels. In the third step 1306 a pick and place automatic component placement (pick-and-place) device such as that known to the art for printed circuit boards is used to place the processor and bypass capacitors to control the actuators on each segment. These are reflow soldered to the array in the same way that printed circuit board soldering is done.

In the fourth step 1308 a pick and place machine attaches electro conductive actuator strips with conductive adhesive, available to the industry, and allows the adhesive a few seconds to set. In the fifth and final step 1310, the pick and place machine now places a connector or battery and radio circuit on one segment of the array is used to control the rest of the array. This element may be of greater size to accommodate these additional components and may provide for array mounting to an application vehicle such as that shown on 8 of FIG. 1 or 604 of FIG. 6.

### Functions & Theory of Operation

Precision Exploration and Intervention

The present invention discloses a microscopic, mobile, and controllable version of an Array Element Mesh System (AEMS), adapted for use in precision-oriented, organic or inorganic applications, typically for exploration, triage, micro-surveillance and monitoring and many diverse intervention tasks, such as microsurgeries, micromanipulation, and transport and delivery of materials.

Micro-scale & Nano-scale Device Dimensions

In some preferred advanced embodiments, the device has microscopic proportions (e.g., sub-1000 nm) so that it may be used in precision-oriented applications (e.g., medical- and/or other small-scale-applications). One with ordinary skill in the art, however, will recognize that the proportions of the device need not be restricted in this manner.

AEMS Devices Adapted for Medical & Other Precision-Control Applications

The small-form-factor version of the AEMS, is based on the original AEMS concept, but further incorporates one or more of locomotion means and/or “course-awareness” of its path in transit; and/or “course direction” to control the direction of its path in transit; locomotion means and/or impeller means; navigation or direction finding and reporting means; tools and/or peripheral actuators; internal and/or external control system means for remote monitoring and control of the device; and possibly a hitch means for linking other devices or auxiliaries.

Diverse Implementation

In alternative embodiments, the device is built as an electronic device or as an electromechanical device; as an organic or inorganic device, as a passive or as an active device; and/or as feasible, based on specific user requirements.

Propulsion and Locomotion

The propulsion and locomotion means of the AEMS-based device permit versions to ambulate on predetermined paths or dynamically-determined paths. In one (of many) possible preferred embodiments, the means for propulsion can comprise either traction feet or rollers, which would permit the device, e.g., to squeeze between cells as applicable or needed for various medical repair, replacement, and/or amelioration applications.

Location and Navigation

There are various methods used to be certain about the location of the AEMS. One of the methods uses the concept of “dead reckoning” as implemented in the AEMS (e.g., “move x steps on the x-axis; y steps on the y-axis, z steps on the z-axis”). This allows it to precisely track its movements and/or be precisely tracked. In this way, the device can track its steps. The location where the device enters the target (e.g., a human or animal body part, surgery site, injection location, or other target object) is referred to as the origin location or zero location (place of insertion, injection, embarkation). When the AEMS keeps track of its location, and/or reports its location to an external control system, it becomes easy to control by a doctor or technician, depending on monitoring features deployed.

NB: This is not to be confused with the concept of the AEMS device “zero state”, wherein the AEMS is implemented “flat” (essentially 2D). An “initialization state” can be any initial state (or configuration) of a device (not just flat).

NB: Regarding location and navigation within a body, the doctor can watch and/or listen (using monitors and stethoscopes) to watch the deployed device's progress as it transits a body or other target object. Also simultaneously the device can “count steps” while it proceeds toward any destination inside a target object. Between what the doctor sees and controls, and the device itself “counting steps” and feeding back data (if provisioned to do so), we can calculate where the device is within the target body or object, in X, Y, and Z planes, on an instantaneous basis.

Radiolocation, Using “Triangulation”

Separately, navigation and direction finding using triangulation apparatus is illustrated: the device is inserted (e.g.) into a human to do knee microsurgery. At the kneecap, or the AEMS insertion point (the AEMS zero location), a plurality of static monitoring devices (3 or more) can be deployed on the zero location skin surface. These radiodomes are placed around 360 degrees of an approximate circle or ellipse perimeter. Using 4 radiolocation devices, with (e.g., typically) one transceiver at approximately each 90-degree quadrant, “triangulation” is possible.

The transceivers are skin-surface-deployed monitors that listen for device beacons (radiolocation signals) transmitted by the inserted AEMS device(s). “Skin Surface Monitor Transceivers” triangulate to get a fix on the moving device within the target body or object. The doctor or technician can observe a video display (e.g.) connected to the external control system (if provisioned). The device tracks all steps in terms of x, y, and z axis . . . it knows where it stepped and it knows where it is located, if provisioned accordingly. (NB: 3D tracking is automated, e.g., using x, y, z-axis analysis and/or volumetric analysis)

Also, “navigation” per se is a relative term: Even if the device knows exactly where it is (or knows where it is with reference to known reference points) and the doctor or tech know where it is, the device still might not (yet) be exactly on the work area target objective it is sent to fix or medicate or treat, e.g., repairing a knee tendon. Or the device may be moved into the wrong part of a target work area and require continuing adjustment until the target site is found.

Tool Actuators

The tool actuators (depending on explicit implementation and configuration capabilities) permit ALDs to, perform predetermined or dynamically determined actions. For example, actuator tools may allow the ALD to perform different programmed tasks, such as penetrating the subject's body by piercing and/or cutting; destruction of unwanted cells; or the deployment of replacement cells. Let's say that in our knee operation, the admin system, and the “Bug” (aka, the adapted version of the ALD), and the doctor all agree that the Bug is in the right place to insert some undifferentiated embryonic stem cells into the area of scarring or broken tendons in the subject's knee. The one or more AEMS Bug(s) in there first extend the embedded injector(s) and flow out the stem cells into the area. Let's say there are some obstructions in the area of the site where the new stem cells are to be inserted. The bug will determine it has to clear an area of obstructions (get out the micro scalpel) and machete-away the interfering or dead tissue. Some versions of the bug might have minilasers that can evaporate the target obstruction. Other versions of the bug might have an excavator tool that dredges out some obstruction and processes it for evaporation or exportation.

Control Systems

The administrative and control system means allow ALDs (Auto Locomotive Devices) and LDs (Locomotive Devices) to be optionally controlled either by external control signals and/or by preprogrammed autonomous control signals from an internal microprocessor or microcontroller. For example, an external control person, typically a doctor or medical technician (or in some applications, an “expert system”), can preprogram device movements and actions and monitor ongoing movements and actions, permitting instantaneous course corrections and enroute activities as needed. NB: Use of the terms “ALD”, “LD”, and the phrase “ALD” and/or “LD” are abbreviated “ALD”. When “ALD” is written, it equally means “ALD”, “LD”, and “ALD” and/or “LD”, for brevity sake.]

“Transformation”

Alternatively, the ALD may transform its physical configuration in response to internal control signals. For example the ALD may transform its' shape from that of an insect-like “Bug” into the shape of a serpentine-like “Snake” of very small diameter in comparison to its length . . . e.g., the ALD could make a snake of the cylindrical dimensions of 20 microns by 200 microns (a 1:10 ratio of width to length). In one embodiment of the invention, the administrative and control means may additionally comprise one or more imaging means to detect the position of ALDs on or inside the target subject. In various embodiments of the invention, the imaging means may be computed tomography (CT), magnetic resonance imaging (MRI), or positron emission tomography (PET). In one preferred embodiment, the imaging means comprises the 3D Body Holographic Scanner technology recently developed at Pacific Northwest National Laboratory (PNNL), which is capable of detailed high-speed 3D imaging.

The administrative and control system may alternatively be analogous to a type of 3-D radar, or use existing tactics from in nuclear medicine and the like, such as effectuating the dyeing and/or radioactive tagging of structures inside the body so their progress can be monitored externally. The administrative and control system can also be interconnected into a medical expert system, a developed or increasingly being developed Artificial Intelligence Medical Expert System.

Finally, the hitch means allows an ALD to connect to another ALD. In one embodiment, the hitch may be comprised of micromechanical means physically linking one ALD to another. In an alternative embodiment, the hitch means may polarize the ALD, such that the “negative” pole of one ALD electromagnetically connects to the “positive” pole of another ALD.

There may be no physical hitching means required or even necessary. It is likely that deployment of multiple individual “Bugs” will for some applications be better than an ALD train, even if preliminarily an ALD train might appear more efficient and effective, or simpler to deploy.

If multiple individual “Bugs” are deployed either at the same time or separately . . . or if they are deployed at fixed and/or at continuous intervals . . . they could be capable of joining or rejoining into train(s) if required (but would not necessarily be joined or rejoined). Ideally, for some applications, much of the intelligence needed to drive and monitor the Bug would be in the admin control system. As implemented, however, depending on application, the distribution of driving and monitoring intelligence can be shared between Bugs and the admin control systems much as feasible.

Using a hitch means, if and where indicated, two or more ALDs may be connected to accomplish one or more target objective(s) undertaken upon or within target work areas (hereinafter referred to as an “ALD train.”). The details of ALD train composition are configured on a case-by-case basis, depending on the work the ALD train is expected to do. ALD trains will generally be mass-designed and mass-assembled, with further customization depending on target requirements. ALD trains can be assembled during their manufacture or intelligently fabricated dynamically. ALD trains can also be chemically assembled, e.g., in a liquid colloidal dispersion. The ALD train can dynamically change its physical dimensions or characteristics, by connecting or disconnecting ALDs, to suit changing mission needs while underway toward a work area or work objective.

External controllers or system administrators or operators, such as a doctor or a technician, may exercise control over the ALD train as a whole, or may elect to control each ALD in the ALD train separately (i.e., detach one or more ALDs in the ALD train, to be deposited by or jettisoned from the train). These examples notwithstanding, ALDs do not need to be linked in a linear fashion, but could be linked in such a way as to create a 2-D or 3-D structure.

The ALD can be configured and implemented in a variety of user-specified embodiments, such that it performs one or more specialized functions. One typical ALD is the “locomotive/navigation ALD” (LN-ALD), which is a self-controlled preprogrammed ALD, typically used to propel itself and one or more ALD trains along a specified or calculated path to reach a target work area or target objective area.

An alternate specialized ALD is the “payload ALD” (P-ALD), which further includes a means for containing and/or supporting extrinsic matter, such that the ALD can transport the extrinsic matter within the target. The means may include a platform, tank, bin, etc. to transport the matter. Alternative structures are anticipated, depending on the nature of the extrinsic matter. The P-ALD can be used, for example, to transport organic matter such as particular molecules, amino acid chains, DNA sequences, etc. The P-ALD may also be used to transport micromechanical equipment, including—but not limited to—cameras, stents, or other devices.

An alternative embodiment of the ALD further includes a “control tail,” (essentially, a guide wire) which is a structural and/or umbilical means for coupling to a larger instrument (such as a surgical tool). The control tail is connected to the administrative 26 and control system means as well as the hitch means of the ALD, so that the ALD or ALD train may be controlled in response to signals from an external source, such as a doctor.

One or more of a scanning tunneling microscope (STM) and/or an atomic force microscope (AFM) can be used to actuate the control tail.

My invention may also be used to import and/or replace and repair damage to intercellular structures, e.g., damage caused to neural pathways in the brain by head trauma. Neural damage vastly affects an individual; as one example, if neural structures in the cerebellum are damaged, the individual's balance, movement, and coordination are affected. For example, to restore or improve “balance”, repairs that may be needed may be establishing or improving “balance” by allowing proprioceptor signals from the inner ear to be transmitted from the inner ear to the balance center in the brain. From the perspective of the balance center in the brain, “balance” in the human body is largely a combined function of gravity, direction, speed, momentum, mass, and other factors. Similarly, the combination of balance, movement, and coordination may be improved if, e.g., the proprioceptor signals and the neural pathways in the brain that they feed can be reconnected and/or amplified. In the case where proprioceptor signals are not getting through to the balance center in the brain, e.g., it may be necessary to repair damaged areas and/or replace signal carrying capabilities in structures which run from the proprioceptors in the inner ear to the balance center in the brain.

The present invention has many possible applications. For example, the invention can be used to import new, healthy mitochondria into cellular arrays. Mitrochondria can be either “healthy” or “unhealthy”, which directly corresponds with the state of health of the subject's body.

When signals are not getting through normal pathways, it is necessary that existing proprioceptor signal relays are bypassed with new signal relays.

In the case of a job to repair a myocardial infarction, the specific ALD trains can be configured with P-ALDs, which can be implemented in target locations, such as the site of a myocardial infarction (scar which forms after some heart attacks). In the process of the perambulation of the ALD to ameliorate the myocardial infarction, the system of the myocardial infarction amelioration includes (1) external monitoring and control instruments operated by a doctor and (2) ALD trains adapted for myocardial infarction remediation. To establish a remediation operation, first the area of the infarction is precisely mapped. Based on the size of the area to be repaired, the number of ALD trains needed for the repair is calculated. A number of ALD trains of predetermined sizes and complements are estimated. The trains are configured to include P-ALDs, which carry: organic scaffold materials and deposition tools for depositing the scaffold on the target remediation site, beating myocytes, and drugs and/or cellular nutrients.

In one embodiment of the invention, the ALD is implemented as an electromechanical device. The administrative and control means may comprise a microprocessor, which can process and execute control signals, and an electronic transceiver, allowing the ALD to receive control signals from other ALDs or external sources. The means for self-propulsion is coupled to the microprocessor, such that it can respond to control signals received from the processor. The means for self-propulsion can receive commands from the microprocessor chip relating to the velocity—both speed and direction—of the means for propulsion; this allows the microprocessor chip to steer the ALD. The ALD also includes a power source, which is connected to the microprocessor and permits the ALD to operate. In various preferred embodiments of the invention, the ALD may receive power through the transceiver in the form of external electromagnetic excitation, may possess a fully self-contained power source, or may consume body fluids of the individual host.

The present invention further discloses methods and systems for controlling and administering the ALD. The administrative system allows doctors, medical assistants, or other individuals to control the velocity of the ALD, as well as tool deployment and tool activation. The administrative system comprises an administrative computer, a means for communication, and one or more ALDs. The administrative computer is a standard computer terminal, allowing individuals means to input commands and view data. The administrative computer is attached to the means for communication, which allows the administrative computer to communicate with the ALDs. In the case of electromechanical ALDs, the means for communication in the administrative ALD may be an electromechanical ALD. There are multiple different ways to communicate with organic devices, some involving transmission of intelligence; others simply using “brute force” methods of communication. In this manner, a doctor may use the administrative computer to control the ALDs.

Applications: Inner Ear Cilia Replacement

Insertion into the AEMS Zero Location

An AEMS adapted for facilitating inner ear surgery is commanded to replace ear cilia into the inner ear. We have the lead LN-ALD and one or more P-ALDs introduced by the doctor into the subject's ear canal. That point of insertion is the “AEMS zero location”. The doctor or tech monitors (and tweaks if needed) the path of progress from the ear canal entry point, the zero location into the inner ear. The device crawls along the ear canal to its target destination within a target object. Alternatively, the doctor injects the devices beneath the inside skin of the inner ear canal.

Malfunctioning Inner Ear Cilia

To illustrate a practical example, the problem is that the target object person's inner ear cilia are malfunctioning—either atrophied or non-responsive due to chronic exposure to loud low-frequency noise (like years in the engine room of a large ship).

Importation, Transport, and Delivery

The invention hauls in a “P-ALD” load of new cilia and/or inner ear goop. The invention could also haul in embryonic stem cells. But in this case the importation of cilia into an inner ear could be re-usage of cilia from the subjects ear which has extra cilia in it due to no damage . . . in this case the Bug would not only be used to INSTALL new cilia into the inner ear . . . but would also be used to EXTRACT a graft of functioning cilia from the subject's own undamaged ear . . . or ear area less damaged.

We have not yet determined whether undifferentiated embryonic stem cells can “make cilia” (or other specialized structures). The limits of embryonic stem cells are currently the subject of the inventor and many other researchers as well.

Possible downstream applications could include one or more of bone marrow transplant and renovation applications; reproductive medicine applications; knee (joint) application; floater retrieval from aqueous humor of the eye or other eye surgery applications; and many others. Actuators in general can also be adapted for other extraction/relocation requirements.

Given the human body's predilection for REJECTING things that it does not recognize, e.g., while it is likely that one's brother or sister can provide a person with bone marrow . . . it is NOT likely that bone marrow can be extracted from another who is not closely related to the subject WITHOUT it being rejected in an unrelated subject's body.

Another concept: a “minitest feature”. It can test the miscibility of the (e.g., imported stem cells or bone marrow) with the body . . . i.e., test that the target work area tissue is COMPATIBLE with the potential-to-be-installed tissue (which is carried by the Bug.) Another concept: the analogy of “Epoxy A and Epoxy B” . . . i.e., there are 2 or more devices, each carries a reactant or tissue or some part of the “recipe” needed for the target objective. Chemical reactions can be effected by combining multiple tissues and reactants or chemicals, at the target work area, with the sequential timing as needed to effectuate the objective, or accomplish the mission.

### Manufacturing the Apparatus

### Overview of Manufacturing the Apparatus of the Invention

For this example, the entire manufacturing and assembly process takes place on a computer-controlled high-resolution XYZ platform with a ˜1 μm positioning accuracy on the Z-pitch (see First Step). These procedures are appropriate for μmanufacturing a wide range of device sizes. Refer also to FIG. 13.

The following is a listing of the basic steps needed to produce preferred embodiments:
1. Manufacture the body of the device.
2. Install the propulsion system.
3. Install sensors/actuators, tools, meds for delivery, etc.
4. Install a system for monitoring, controlling, tracking, reporting progress of device build up/down (and disposition after deployment).
5. Fifth Step: Final trim and clean-up of manufactured apparatus

The preferred embodiment of the device might include devices which are large enough to be manufactured by means of existing automation equipment or by manual assembly, however, smaller embodiment version, including in-vitro medical devices, require the use of manufacturing dimensions in the microscopic range using microscopic techniques or nanotechnology where appropriate. The dimensions of such devices will be measured in micrometers and are too small for manual or “conventional” assembly methods.

1. First Step: Manufacturing the Body of the Device

The entire device is built upon an XYZ plate similar to those used by precision plotters and 3D printers. Here, we use a URM-HP 301: it is a Micro-Stereolithography printer (available from Unirapid Inc., Misato City, Japan). See: unirapid.com/eng_micro_rp.html (rendered in Japanese but it can be translated by Google or other translator facility).

For larger-sized versions of preferred embodiments using wall thicknesses greater than 15 μm, a mass production manufacturing version of the device, supra, is used. That product is the Yunirapitto III (Unirapid III).

An American source of information about the larger-scale 3-D printer is available at: www.3dprinter.net/japan-company-races-to-ultra-precise-desktop-3d-printing NB: The wall thicknesses produced by that much-less-expensive device are larger (>100 μm).

NB: The higher-resolution 3D printer--the URM-HP 301, Micro-Stereo-lithography printer—can also be used to make the smaller-form factor versions of the apparatus of the invention. It is capable of creating objects using a laser beam of ˜7 μm beamwidth. The positioning accuracy of the Z-pitch is ˜1 μm per layer. Objects can be built with a wall thickness of a minimum of ˜15 μm (twice the laser beamwidth).

Several high resolution fabrication materials are available, some with good biocompatibility, including colorless acrylic resin, resin reinforced with a hard filler, photonic amorphous diamond material, and others currently in development. The CAD (computer automated design) commands to the 3D printer are often created by 3-D object modeling software such as SolidWorks (or equivalent-function applications). SolidWorks available from: Dassault Systems SolidWorks Corp. See: www.solidworks.com

The device can be manufactured and held in place by a small standoff from its mounting base attached to the XYZ platform. This serves to immobilize the device while it is being assembled. This standoff will be cut off at the end of the manufacturing process by passing the device by a sharp razor or trimmer, or a laser cutter or the like.

2. Second Step: Power and Propulsion, Including Installation

The supply of energy needed to drive the present invention depends upon the version's size and its applications. For short-depth (or any depth) penetration of the body, a tether can optionally be provided which can directly provide power. Optionally, a tether can be also used to withdraw the apparatus at the end of its task. However, for greater depth and greater freedom of motion within the body, it is necessary for the device to receive power or store it, at least for any feasible untethered application (if any). NB: It is obvious that “feasibility” is relative: e.g., what may be feasible tolerances and operating parameters for a structural engineering application may not be feasible or reasonably prudent or “safe” for a neurosurgery app. Motive energy can be obtained with inducted electrical power and/or by means of at least one rechargeable or replaceable “micro-battery” or other fuel source. Given the state of the art, we can use (1) a battery containing fully-insulated battery components and/or we can use (2) biologically-compatible compounds and elements as power mechanisms.

Power Options

There are basically three methods for providing power to in vivo devices:
a. A wire-connected system using a tether containing conductors.
b. A wireless system that receives power that is transmitted from outside of the body.
c. A storage system that carries its own energy by means of carrying a charged battery or by deriving power from body fluids.

From these options, two preferred embodiments described. (Other options are available)

For easily penetrated areas, such as the outer ear or sinus passages, a wired system can carry out most medical tasks. It has the advantage of enabling extraction by means of the tether in case the device gets stuck. It is not ideally suited, however, for deeper penetration or where body scanning is concurrently performed. Also, the exit path must be exactly the reverse of the entrance path, limiting exploration. Alternatively, extant guide-wires (e.g., as for balloon angioplasty and the like) can be adapted to carry along and/or discharge the apparatus of the invention when/where it is needed to be delivered in vivo.

For more extensive exploration and treatment within the body a wireless version may be preferred, with or without a tether. In this case, a wireless antenna receives a signal originating from an outside source, such as in inductive transmitter or the oscillating electromagnet field of a scanner: There is variability in the received power level depending upon device orientation or local body electromagnetic absorption. In order to maintain a consistent internal power supply for the processor, sensors and actuators, it will be necessary to supply a capacitor and/or biocompatible battery source within the device. These components may reside on the same substrate as that of the propulsion system and sensor array, described below.

A preferred manufacturing technique is print the circuit on a polymer substrate and then insert it into the body while it resides on the XYZ plate as described in Step 1, above. An alternative assembly method is to place the completed electronics circuit in a tube that can absorb focused heat directly in the Stereo Lithographic (SLA) fluid co-located in the place where the focused light source will be directed to construct the body. In this case, the SLA material will be hardened around the tube, sealing the electronics within the body in one step.

Propulsion

For small- and meso-scale form factors amenable to manual or conventional automated manufacturing, available materials can be cut or punched to required shapes. Actuators that propel the device may be constructed from either triangular plates or flexible film made of off-the-shelf materials. They are rotated along the hinged or bending axes by extant EAP (ElectroActive Polymer) films/sheets, e. g., polyprrole.

For micro and nano-sized form factors, the apparatus of the present invention can be constructed using technologies that as described in MIT publication: “Microfabricating Conjugated Polymer Actuators”. See: http://www-mtl.mit.edu/researchgroups/mems-salon/yawen Microfabricating conjugated polymer actuators.pdf

In order to avoid the problems of slow diffusion rate and swelling in width in nano-scale devices, the MIT Department of Chemistry has investigated the use of molecular mechanisms, which utilize a dimensional change of a single polymer chain to expand or contract the electrochemically-controlled applied actuator membrane. This is described, at: http://dspace.mit.edu/handle/1721.1/41554 Thus, propulsion mechanisms described in the specification can be manufactured anywhere in the size range from visible- to nano-scale devices as needed/required for diverse applications.

3. Third Step—Manufacturing the Sensor Array

Installing Sensors for sensing observable phenomena on the meso-, micro, and nano-scale

Using micro- and nano-technology devices, we can integrate array(s) of small-form factor sensors for in-vivo, in-vitro, and/or ex-vivo applications. Such sensors (depending on application needs) can allow analysis and sensing of many chemicals, types of mechanical motion, and/or pressure/force sensing. This work is ongoing at several leading universities, e.g., in the University of British Columbia, Canada and Massachusetts Institute of Technology. The next level of sensor analysis . . . will involve the inclusion of a confocal imaging engine using MOEMS (i. e., micro-optical-electro-mechanical”) technology which can produce microscopic video images from an extremely small device. Current research is progressing to couple this micro-imaging with Raman Spectroscopy using MEMS technology in order to enable spectrographic analysis by a non-intrusive probe which can be incorporated in the current invention. Currently, the development of such devices is ongoing at MiNa (Microsystems & Nanotechnology) group at University of British Columbia, Canada.

NB: Please see also: http://www.mina.ubc.ca/project magnetically-driven-micro-confocal-imaging-system

4. Fourth Step—Processor and Wireless Communications

Although Since the year 2000, a number of individual nanoscale computing devices (e.g. wires, logic gates and memory cells) have been demonstrated, the smallest microprocessors and radio circuits at the date of the specification of the present invention were mass produced for wireless smartcards. A simple wireless smartcard microprocessor with short-range NFC communications could be produced at i.5 mm square with 45 nm technology in 2006 and 33 nm in 2008. Thus, the specification for the invention was described with available technology for very small, but not nano-scale device construction. Knowing that there was a continuously reducing scale of microprocessor technology enabled the same invention to be reduced to nano-scale in the immediate future makes the eventual miniaturization practically unlimited as described in http://royal.pingdom.com/2012/02/29/the-single-atom-transistor-is-here-the-amazing-evolution-of-microprocessors-infoqraphic/.

Such computing can be done reliably despite randomizing molecular and atomic effects as described in: “Reliable Computing at the Nanoscale”

NB: Please see: http://cs.brown.edu/˜eerac/papers/phd_thesis.pdf

Note that wireless communication with the device is very short range and the receiver gain at the controlling station is virtually unlimited. The frequency is in the 2-10 Ghz range to enable the use of a correspondingly small antenna which essentially establishes the length of the current device.

5. Fifth Step—Test, Standoff Cutting and Packaging

The final step is to perform the appropriate test procedure and then move the XYZ plate from the position of Steps 2-4 to the position of Step 5 such that the device passes past a cutoff blade that severs the standoff between the bottom of the device and the underlying plate that was created in Step 1. This enables the device to be dropped into the shipping package.

### Nanotechnology Products and Applications: Product Details

Product: EcoSphere

Manufacturer: EcoSynthetix

Product page: (external link′)

Product Sector: Chemicals→Green Chemistry

Details:

EcoSphere® is a new bio-based material as an alternative to petroleum-based latex. It can broadly substitute for oil-based latex in many applications, e.g., paper and paperboard, architectural coatings, carpet backing, engineered wood products, insulation/roofing, cosmetics, textiles, nonwovens and drilling fluids.

Much like the Intel microchip powers different brands of computer, the “EcoSphere Inside” concept enables partners to power their own sustainability initiatives. In the paper industry, EcoSphere biolatex binders are a new family of commercially available products for manufacturing coated paper and paperboard, providing a traditional industry an alternative to non-renewable petroleum chemicals. These products result from the transformation of annually renewable crop resources via the company's patented processes into a dry biopolymer nanoparticle agglomerate powder that can be used dry or pre-dispersed in water.

This family of EcoSphere products exhibit excellent ultra-high shear rheology, shear-thinning behavior similar to petrochemical-based colloids such as carboxylated styrene butadiene (SB), styrene acrylate (SA) and polyvinyl acetate (PVAc) latex.

See: http://www.nanowerk.com/products/product.php?id=170#ixzz2XixzOBCs

I claim:

1. An AEMS apparatus adapted for precision-control applications, comprising: at least one substrate; at least one processor, at least one transceiver coupled to said at least one processor; at least one of an actuator tool and a means for transporting a payload; and at least one means for pointing said AEMS device toward a target destination.

2. The apparatus of claim 1, further including at least one of a propulsion means and a locomotion means.

3. The apparatus of claim 1, further including at least one of a power source and a power supply.

4. The apparatus of claim 1, wherein said at least one substrate is comprised of a plurality of array elements integrated to form an AEMS further comprising the body of said apparatus.

5. The apparatus of claim 1, wherein said at least one processor includes instructions for at least one of (but not limited to):
(i) determining apparatus position and determining and extrapolating position changes over time when operating within a target object;
(ii) determining at least one of the relative position of said apparatus and the absolute position of said apparatus when compared to at least one reference point determining distance to a destination within said target object;
(iii) determining instantaneous best path to said destination;
(iv) pointing said apparatus toward said destination within said target object;
(v) communicating to and from at least one external control system; and
(vi) controlling operation of said apparatus including operation of at feast one array element in said AEMS in order to do work and in order to direct movement of said apparatus toward at least one said destination located within said target object.

6. The apparatus of claim 1, wherein said transceiver includes means for communicating signals between said apparatus and at least one external control system.

7. The apparatus of claim 1, wherein at least one of a propulsion means and a locomotion means is co-located with said external control system.

8. The apparatus of claim 1, wherein said power supply is external to said apparatus and comprises a source of electrical energy adapted for transmission to said apparatus, and wherein the locomotion and propulsion of said apparatus is precisely controlled by varying the strength and directionality of said electrical energy transmitted to said apparatus.

9. The apparatus of claim 1, further comprising at least one interconnector for connecting to and disconnecting from at least one other AEMS apparatus.

10. An external control system for monitoring, tracking, and controlling propulsion, locomotion, and operation of an AEMS apparatus, comprising: at least one processor including instructions for monitoring, tracking, controlling, initiating, launching, executing, and terminating propulsion, locomotion, and operation of said AEMS apparatus; a transceiver for communicating between said external control system and said AEMS apparatus; and at least one of a monitor connected to at least one computer.

11. The external control system of claim 10, further comprising a linking and tethering system for at least one of communicating with and for controlling locomotion of said AEMS apparatus.

12. The apparatus of claim 2, wherein said at least one propulsion means comprises at least one of traction feet and rollers.

13. The apparatus of claim 12, wherein said traction feet further approximately comprise alternately-facing locomotive feet for moving said ALD apparatus toward at least one of a target work area and a 3D target object and a target destination therewithin, and wherein said locomotive feet are at least one of retractable within said ALD body of said ALD apparatus and extendable out of said ALD body of said ALD apparatus.

14. The apparatus of claim 1, wherein said at least one actuator tool comprises a scalpel actuator tool for piercing and cutting info a target work area.

15. The apparatus of claim 1, wherein said at least one actuator tool comprises a disruptor actuator tool; wherein said disruptor actuator tool is adapted for destroying human organic cellular material; and wherein said disruptor actuator tool is also adapted for destroying animal cells.

16. The apparatus of claim 1, wherein said at least one actuator tool comprises a payload actuator tool; wherein said at least one payload actuator tool is adapted for deploying replacement animal cells.

17. The apparatus of claim 12, wherein said at least one payload actuator tool is also adapted for deploying replacement animal cells comprising replacement human myocytes.

18. The apparatus of claim 12, wherein said at least one payload actuator tool is also adapted for deploying replacement animal cells comprising replacement rodent myocytes.

19. The apparatus of claim 1, wherein said power source is charged through external electromagnetic excitation.

20. The apparatus of claim 1, wherein said power source is a self-contained battery.

21. The ALD of claim 1, wherein said power source is charged through consumption of absorbable bodily fluids integral to an animal subject.

22. The administrative system of claim 22, wherein said wire-connected interface means further comprises at least one control tail coupled into at least one control tail fitting.

23. An ALD train assembly system for interconnecting a plurality of AEMS-ALDs into at least one ALD train, comprising:
(i) instrument means for staging and organizing component ALDs prior to assembly of said at least one ALD train;
(ii) means for selecting and organizing assembly of said component ALDs including at least one of selecting and organizing of an LN-ALD component and a Payload-ALD component;
(iii) means for further coupling said component ALDs together to assemble at least one ALD train further comprising at least two coupling devices disposed upon at least two control tail fittings of each said component ALDs;
(iv) means for communicating between said component ALDs comprising said assembled ALD train; and
(v) further including means for communicating between said ALD train and at least one external control means.

24. The administrative system of claim 23, wherein the means for imaging is at least one of computed tomography (CT) technology; magnetic resonance imaging (MRI) technology; positron emission tomography (PET) technology; and 3D Body Holographic Scanner technology.

25. An array element apparatus for interconnecting and communicating with at least one adjacent array element, comprising a processor having at least one input/output/control communication interface, and at least one of a joint position actuator interface, a joint position sensor/encoder interface, a strain gauge interface, and a digital-to-analog interface, wherein:
said processor is embedded and coupled to at least one flexible interconnector substrate;
an input/output/control communication line and at least one of a joint position actuator is deployed therewithin and coupled thereto;
a joint position sensor/encoder, a strain gauge, and a digital-to-analog converter are coupled into said processor;
and wherein:
said substrate is further adapted for communicating, coupling, and providing power and continuity between the processor of said array element and the processor of said at least one adjacent array element; and said substrate further includes at least one of a joint position actuator, a joint position sensor/encoder, a strain gauge, and a digital-to-analog converter coupled thereto.

26. The apparatus of claim 25, further comprising at least one power source.

27. The apparatus of claim 26, wherein said power source further comprises but is not limited to at least one of an electrical source, an electromagnetic source, a magnetic induction source, an electrostatic source, a chemical source, a photonic source, and a radiant source.

28. The apparatus of claim 25, further comprising at least one local network coupled into said processor and into the processor of said at least one adjacent array element, wherein said local network is further adapted to exchange data between and among said processor, the processor of said at least one adjacent array element, and any processor connected to said substrate.

29. The Apparatus of claim 25, wherein said flexible interconnector substrate is comprised of at least one flexible structural material having at least two dimensions, and wherein said substrate is flexible in three dimensions.

30. The Apparatus of claim 29, wherein said substrate is further adapted to include additional components disposed onto or within said array element apparatus.

31. The apparatus of claim 25, wherein more than one of said array element apparatus is adapted for interconnection into additional adjacent array elements to comprise in combination an array element mesh system.

32. The apparatus of claim 25, wherein said array element apparatus is coupled to said at least one flexible interconnector substrate at areas of flexure disposed between said array element apparatus and said at least one adjacent array element.

33. The apparatus of claim 25, wherein more than one of said array element apparatus is adapted for interconnection into additional adjacent array elements to comprise in combination an array element mesh system.

34. The apparatus of claim 25, wherein said array element apparatus is coupled to said at least one flexible interconnector substrate at areas of flexure disposed between said array element apparatus and said at least one adjacent array element.

35. The apparatus of claim 25, wherein the surface area of said array element apparatus is smaller than the surface area of said at least one flexible interconnector substrate.

36. The apparatus of claim 25, wherein the surface area of said array element apparatus is approximately equal to the surface area of said at least one flexible interconnector substrate.

37. The apparatus of claim 25,
wherein said input/output/control communication line and said at least one of a joint position actuator, a joint position sensor/encoder, a strain gauge, and a digital-to-analog converter coupled to said at least one processor are further coupled to said at least one flexible interconnector substrate at areas of flexure disposed between said array element apparatus and said at least one adjacent array element;
wherein said input/output/control communication line and said at least one of a joint position actuator, a joint position sensor/encoder, a strain gauge, and a digital-to-analog converter are adapted to respond to at least one command;
wherein said input/output/control communication line and said at least one of a joint position actuator, a joint position sensor/encoder, a strain gauge, and a digital-to-analog converter are further adapted for sensing and reporting position and movement of said at least one adjacent array element in relation to said array element apparatus, and
wherein said input/output/control communication line and said at least one of a joint position actuator, a joint position sensor/encoder, a strain gauge, and a digital-to-analog converter are further adapted to store data in and retrieve data from the memory of said processor.

38. The apparatus of claim 37, wherein said at least one command is issued by one of a processor internal to said array element apparatus and a processor external to said array element apparatus; and wherein said at least one command further comprises but is not limited to at least one of: (1) a sense position command, (2) a report position command, (3) a learn position command, (4) a move position command, and (5) a set position command.

39. The apparatus of claim 37,
wherein said at least one joint position actuator is adapted to respond to said at least one command; and
wherein said joint position actuator is further adapted to respond by moving at least one of said adjacent array elements from a first position to a second position in relation to said array element apparatus.

40. The apparatus of claim 28, wherein said at least one local network is adapted to exchange data between said processor, and at least one processor of said adjacent array element, any processor connected to said substrate, and at least one external processor coupled to an external system.

41. The apparatus of claim 40, wherein said at least one local network is further adapted to exchange data between and among (1) said processor, (2) said processor of said adjacent array element, (3) said any processor connected to said substrate, (4) the processor of at least one base array element or at least one supervisory processor, and (5) at least one external processor coupled to an external system.

42. The apparatus of claim 28, wherein said at least one local network includes at least one conductive wired LAN circuit coupled to said substrate; and
wherein said at least one local network is coupled to the network transceiver included within at least one of (1) said processor, (2) said processor of said adjacent array element, (3) said any processor connected to said substrate, (4) said processor of said at least one base array element or said at least one supervisory processor, and (5) said at least one external processor coupled to an external system.

43. The apparatus of claim 5, wherein said at least one local network includes at least one wireless WPAN circuit coupled to said substrate; and wherein said at least one local network is coupled to the network transceiver included within at least one of (1) said processor, (2) said processor of said adjacent array element, (3) said any processor connected to said substrate, (4) said processor of said at least one base array element or said at least one supervisory processor, and (5) said at least one external processor coupled to an external system.

44. The apparatus of claim 5, wherein said local network is further adapted to send and receive joint position information between and among (1) said processor, (2) said processor of said adjacent array element, (3) said any processor connected to said substrate, (4) said processor of said at least one base array element or said at least one supervisory processor, and (5) said at least one external processor coupled to an external system.

45. The apparatus of claim 37, wherein said at least one command is provided in parametric form and is further provided according to a predetermined frequency.

46. An array element mesh system comprising:
a variably configurable robotic surface, further comprising a plurality of interconnected array elements coupled to at least one flexible interconnector substrate;
each of said plurality of interconnected array elements further comprising at least one of: a processor, a supervisory processor, a joint position sensor, a joint position actuator, a strain gauge, a digital-to-analog converter, and an input/output/control line including a communication link;
at least one local network;
and
at least one network connection to at least one of:
(i) a processor,
(ii) the processor of at least one adjacent array element,
(iii) any additional processor connected to said substrate,
(iv) the processor of at least one base array element or at least one supervisory processor, and
(v) at least one external processor coupled to an external system; and
(vi) software instructions executing within at least one of said processor, the processor of said at least one adjacent array element, said any additional processor connected to said substrate, said processor of said at least one base array element or said at least one supervisory processor, and said at least one external processor coupled to an external system for issuing commands to at least one of said plurality of interconnected array elements.

47. The system of claim 46, wherein said system is further adapted for at least one of but is not limited to: (1) sampling and simulating a target 3D object, and (2) sensing positions of at least one array element in relation to at least one other array element, and (3) learning 3D shape data from said 3D object, and (4) optionally playing back learned 3D shape data in a 2D image display or a 3D simulation, and (5) optionally replicating at least one function of said 3D object and/or replicating the shape of said 3D object if said system is capable of replication thereof and if said system is configured for replication.

48. The system of claim 46, further comprising a power source, wherein said power source further comprises but is not limited to at least one of an electrical source and an electromagnetic source and a magnetic induction source and a electrostatic source and chemical source and a photonic source and a radiant source.

49. The system of claim 46, wherein said local network is adapted for coupling said processor and said supervisory processor to at least one other processor, and wherein said at least one other processor is at least one of included within said system and external to said system.

50. The system of claim 46, wherein said software instructions provide programming steps and commands to do at least one of:
(1) sense and report joint position status data characteristic to a sampled 3D object;
(2) move at least one array element from a first position to a second position to conform said system to the surface of said 3D object;
(3) sense and report changed joint position status data after moving said at least one array element;
(4) learn joint position status data characteristic to said 3D object;
(5) playback at feast one joint position characteristic to said 3D object; and
(6) provide at least one of 2D image display and a 3D simulation and a 3D replication of said 3D object if said system is capable thereof.
